(19) United States
(12) Patent Application Publication
(10) Pub. No.: US 2023/0011536 A1
(43) Pub. Date: Jan. 12, 2023

(54) MESENCHYMAL STEM CELL DERIVED EXTRACELLULAR VESICLES LOADED WITH AT LEAST ONE PHOTOSENSITIZER AND USES THEREOF FOR THE TREATMENT OF PERITONEAL CARCINOMATOSIS

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); ASSISTANCE PUBLIQUE-HÔPITAUX DE PARIS (AHPH), Paris (FR); UNIVERSITÉ PARIS CITÉ, Paris (FR)

(72) Inventors: Marc POCARD, Paris (FR); Florence GAZEAU, Paris Cedex 13 (FR); Iris MARANGON, Paris Cedex 13 (FR); Amandine PINTO, Paris Cedex 10 (FR); Amanda SILVA, Paris Cedex 13 (FR); Max PIFFOUX, Villejuif (FR); Claire WILHELM, Paris Cedex 13 (FR)

(21) Appl. No.: 17/777,740

(22) PCT Filed: Nov. 20, 2020
(86) PCT No.: PCT/EP2020/082891

§ 371 Date: May 18, 2022

(30) Foreign Application Priority Data
Nov. 21, 2019 (EP) 19306495.3

(51) Int. Cl. A61K 35/28; A61K 31/409; A61K 41/00; A61P 35/00; C12N 5/0775

(52) U.S. Cl. CPC A61K 35/28; A61K 31/409; A61K 41/0071; A61P 35/00; C12N 5/0663

(57) ABSTRACT

Several gastrointestinal and gynecological malignancies have the potential to disseminate and grow in the peritoneal cavity. The occurrence of peritoneal carcinomatosis (PC) has been shown to significantly decrease overall survival in patients. Treatment of residual microscopic disease remains a challenge with new anticancer modalities development. Now, the inventors propose an innovative therapeutic management of peritoneal carcinomatosis (PC) that is bio-inspired and tumor-targeted by engineering MSC-derived EVs to encapsulate a photosensitizer (mTHPC) for improved photodynamic therapy efficiency and safety. In this work, the inventors first evaluated the biodistribution of EVs-mTHPC in a murine PC model and highlighted superior accumulation of mTHPC in the tumor compared to other mTHPC formulations (free drug and liposomal one (Foslip®). The effectiveness of PDT mediated by mTHPC vectorized in EVs has then been evaluated in PC. In accordance with pharmacokinetics, the results revealed both an enhanced light-induced therapeutic efficiency in terms of tumoral cytotoxicity, safety for surrounding tissue after laser irradiation, immunomodulation and improved survival time. Thus, the present invention relates to mesenchymal stem cell derived extracellular vesicles loaded with at least one photosensitizer and uses thereof for the treatment of peritoneal carcinomatosis.

### FIELD OF THE INVENTION

The present invention is in the field of medicine, in particular oncology.

### BACKGROUND OF THE INVENTION

Peritoneal carcinomatosis (PC) describes the dissemination of cancer deposits within the peritoneal cavity. The therapeutic modalities and prognosis depend on the origin of the primary cancer.

Peritoneal carcinomatosis secondary to colorectal cancer have a poor prognosis, about 16 months with chemotherapy treatment [Franko]. The development of combined treatment involving cytoreductive surgery (CRS) and hyperthermic intraperitoneal chemotherapy (HIPEC) permitted to improve survival of these patients [Elias Lefevre 2009, Elias Gilly 2010, Sugarbaker 2012] with a median survival of more than 40 months [Elias Lefevre 2009, Elias Gilly 2010, Chua 2009, Quenet Goéré 2011, Ceelen 2018]. However the recent result of the first phase III trial (PRODIGE 7) comparing CRS alone with CRS combined with HIPEC using oxaliplatin, failed to demonstrate an overall survival advantage in the HIPEC arm [Quenet 2018]. Post-operative morbidity rate, with a 60 day complication rate significantly higher (24.1% vs 13.6%, p=0.03) for patients treated with HIPEC [Quenet 2018] could explain the lack of benefit in the Prodige 7 trial. Treatment of residual microscopic disease remains a challenge with new anticancer modalities development.

Photodynamic therapy (PDT) is a recent therapeutic anti-cancer modality in the clinical care [Diamond 1972]. PDT permits to selective destruction of cancerous tissue after accumulation of a photosensitizer (PS) in cancer cells, more rapidly than nonmalignant tissue. Upon PS activation with a particular wavelength illumination, there is a transfer of its excited-state energy to surrounding oxygen. This results in the production of reactive oxygen species (ROS), such as singlet oxygen, which will induce cell death. There are two mechanism of cell death: direct mechanism (necrosis and apoptosis) and indirect mechanism (microvascular damage, and antitumor immune responses) [Dougherty 1998, Cengel 2007, Castano]. Tochner et al. [Tochner 1985] first in 1985, investigated the use of PDT in PC murine model with a high cure rate of 85%. With the arrival of second generation of PS, especially temopophin—mTHPC® (meta(tetrahydroxyphenyl)chlorin), others authors interested in the effectiveness of PDT for PC treatment [Pinto, 2018]. mTHPC is considered to be one of the most potent and selective second-generation PS in present use [Senge2015]. Real limit is the toxicity, particularly induced by highly aggregated of mTHPC in biological medium due to high lipophilicity, and the lack of specificity of PS for tumor tissue. To solve this problem, the ultimate generation of PS was developed, consisting in targeted PS [Azais 2016 et 2017, Yokoyama 2016]. Preclinical studies with new PS showed an important tumoral biodistribution in peritoneal carcinomatosis model (ratio 9.6 vs normal tissue in ovarian model [Azais]). This treatment demonstrated a significant survival advantage too in rats models (35.5 vs 52.5 days for cytoreductive surgery vs cytoreductive surgery+PDT, p<0.005 [Yokoyama]). Two formulations of liposomal nanovectorization of mTHPC are currently available: Foslip® and Fospeg®. Water-soluble liposomes enable selective accumulation in tumor tissue and thereby improve the drug selectivity and PDT efficacy. In colorectal tumor model, Foslip® demonstrated a rapid biodistribution and clearance from the bloodstream with an average tumor-to-muscle and the tumor-to-skin selectivity (6.6 and 2 respectively at 2 and 8 h after injection) [Svensson 2007]. With Fospeg®, a higher plasma peak concentration, a longer circulation time and a better tumor-to-skin ratio than those of Foslip, were shown [Xie H 2015]. However, limited penetration of liposomal mTHPC in tumor tissue limited mTHPC-based liposomal applications in the clinical practice [Lassalle 2009, Robella 2019, Sugarbaker 2019, Mikolajczyk 2018].

Development of new nanovectorization of mTHPC to improve tumor selectivity in PC treatment interest a lot of teams. Due to their tropism to the tumor niche, mesenchymental stem cells (MSC) are promising vectors for the delivery of antitumoral treatment [Chulpanova 2018]. Moreover, Extracellular Vesicles (EVs) appeared like attractive candidates for efficient drug delivery [Camussi 2011-Silva 2013]. MSC derived membrane microvesicle (EVs-MSC) had an important role in intercellular communication, and they are considered as a new drug delivery vector via the loading of these structures with therapeutic agents [Chulpanova 2018]. They constitute a bio-camouflaged delivery system for exogenous therapeutic agents [Piffoux 2018]. Contrary to synthetic nanovectors as liposomes, EVs-MSC appears like a biogenic drug delivery vehicles, to deliver therapeutic agents such as therapeutic miRNA and anti-cancer agents [Silva 2015, Moore 2017, Chulpanova 2018]. EVs are considered to be natural nanocarriers with improved biocompatibility, which is advantageous for clinical tranlation [Syn 2017]. Similarly to liposomes, the EV membrane is essentially a lipid bilayer protecting the payload from degradation, but with natural targeting properties and relative stability in blood circulation[Vader 2016]. In multicellular tumor spheroids (in vitro 3D model), better mTHPC accumulation and penetration (up to 100 mm) were observed for EVs-mTHPC compared with liposomal mTHPC [Millard 2018]. This first analysis permitted to show that EVs should be considered as perspective nanocarriers for mTHPC mediated PDT [Millard 2018].

### SUMMARY OF THE INVENTION

As defined by the claims, the present invention relates to mesenchymal stem cell derived extracellular vesicles loaded with at least one photosensitizer and uses thereof for the treatment of peritoneal carcinomatosis.

### DETAILED DESCRIPTION OF THE INVENTION

Bio-camouflaged and bio-inspired approaches are currently very attractive strategies in the emerging nanomedicine landscape for the delivery of drugs. In particular, extracellular vesicles (EVs) could represent a drug delivery vehicle of choice considering their endogenous properties of stability in blood circulation, immunotolerance and capacity to facilitate cell entry. In the specific context of tumor therapy, mesenchymal stem cells (MSC) present inherent tumor-trophic properties whose MSC-derived EV could inherit, which allow them to serve as vehicles for targeted therapy to isolated tumors and metastatic disease. From this perspective, the inventors propose an innovative therapeutic management of peritoneal carcinomatosis (PC) that is bio-inspired and tumor-targeted by engineering MSC-derived EVs to encapsulate a photosensitizer (mTHPC) for improved photodynamic therapy efficiency and safety. For this purpose, a pioneering strategy for high yield and large scale EV-production was used following producer cell loading with the mTHPC photosensitizer. In this work, the inventors first evaluated the biodistribution of EVs-mTHPC in a murine PC model and highlighted superior accumulation of mTHPC in the tumor compared to other mTHPC formulations (free drug and liposomal one (Foslip®). The effectiveness of PDT mediated by mTHPC vectorized in EVs has then been evaluated in PC. In accordance with pharmacokinetics, the results revealed both an enhanced light-induced therapeutic efficiency in terms of tumoral cytotoxicity, safety for surrounding tissue after laser irradiation, immunomodulation and improved survival time. This study provides insight into the benefits of EVs as biocamouflaged nanovector for improved PDT accuracy, control and targeting and paves the way to overcome current limitations of ongoing strategies for PC management, notably, resistance and non-specificity.

Thus, the first object of the present invention relates to an isolated mesenchymal stem cell derived extracellular vesicle loaded with at least one photosensitizer.

As used, herein, the term “mesenchymal stem cell” or “MSC” has its general meaning in the art and refers to multipotent stromal cells that can differentiate into a variety of cell types, including: osteoblasts (bone cells), chondrocytes (cartilage cells), myocytes (muscle cells) and adipocytes (fat cells) (See for example Wang, Stem Cells 2004; 22(7); 1330-7; McElreavey; 1991 Biochem Soc Trans (1); 29s; Takechi, Placenta 1993 March/April; 14 (2); 235-45; Takechi, 1993; Kobayashi; Early Human Development; 1998; Jul. 10; 51 (3); 223-33; Yen; Stem Cells; 2005; 23 (1) 3-9.) These cells may be defined phenotypically by gene or protein expression. These cells have been characterized to express (and thus be positive for) one or more of CD13, CD29, CD44, CD49a, b, c, e, f, CD51, CD54, CD58, CD71, CD73, CD90, CD102, CD105, CD106, CDw119, CD120a, CD120b, CD123, CD124, CD126, CD127, CD140a, CD166, P75, TGF-bIR, TGF-bIIR, HLA-A, B, C, SSEA-3, SSEA-4, D7 and PD-L 1. These cells have also been characterized as not expressing (and thus being negative for) CD3, CD5, CD6, CD9, CD10, CD11a, CD14, CD15, CD18, CD21, CD25, CD31, CD34, CD36, CD38, CD45, CD49d, CD50, CD62E, L, S, CD80, CD86, CD95, CD117, CD133, SSEA-1, and ABO. MSCs can be isolated using methods known in the art, e.g., from bone marrow mononuclear cells, umbilical cord blood, adipose tissue, placental tissue, based on their adherence to tissue culture plastic. For example, MSCs can be isolated from commercially available bone marrow aspirates. Enrichment of MSCs within a population of cells can be achieved using methods known in the art including but not limited to FACS.

As used herein, the term “extracellular vesicle” refers to a cell-derived vesicle comprising a membrane that encloses an internal space. Extracellular vesicles comprise all membrane-bound vesicles that have a smaller diameter than the cell from which they are derived. Generally, extracellular vesicles range in diameter from 50 nm to 1000 nm, and can comprise various macromolecular cargo either within the internal space, displayed on the external surface of the extracellular vesicle, and/or spanning the membrane. In some embodiments, the diameter of the MSC-EV according to the present invention is about 175 nm. Thus, as used herein, the term “mesenchymal stem cell derived extracellular vesicle” or “MSC-EV” refers to an extracellular vesicle originated from mesenchymal stem cells. Thus, the MSC-EVs of the present invention are identifiable by the detectable presence of a particular surface epitope or combination of surface epitopes, and/or by the absence of particular surface epitopes. For example, the present enriched population of MSC-EVs are characterized by the presence of vesicle surface detectable levels of the epitope CD63 (i.e., CD63(+)), CD81 (i.e., CD81+), and CD9 (i.e., CD9⁺).

As used herein, the terms “isolated,” “isolating,” “purified,” “purifying,” “enriched,” and “enriching,” as used herein with respect to cells, means that the MSC-EVs at some point in time were separated, purified, and capable of therapeutic use. “Highly purified,” “highly enriched,” and “highly isolated,” when used with respect to said extracellular vesicles, indicates that the cells of interest are at least about 70%, about 75%, about 80%, about 85% about 90% or more of the cells, about 95%, at least 99% pure, at least 99.5% pure, or at least 99.9% pure or more of the cells, and can preferably be about 95% or more of the EVs.

As used herein, the term “photosensitizer” or “PS” has its general leaning in the art and refers to a chemical compound that produces a biological effect upon photoactivation or a biological precursor of a compound that produces a biological effect upon photoactivation. Photosensitizers of the invention can be any known in the art. Typically, the photosensitizer of the present invention has a chemical structure that includes multiple conjugated rings that allow for light absorption and photoactivation, e.g., the photosensitizer can produce singlet oxygen upon absorption of electromagnetic irradiation at the proper energy level and wavelength.

In some embodiments, the photosensitizer is selected among porphyrins and hydroporphyrins. Example of porphyrins and hydroporphyrins include, but are not limited to, Photofrin® RTM (porfimer sodium), hematoporphyrin IX, hematoporphyrin esters, dihematoporphyrin ester, synthetic diporphyrins, O-substituted tetraphenyl porphyrins (picket fence porphyrins), 3,1-meso tetrakis (o-propionamido phenyl) porphyrin, hydroporphyrins, benzoporphyrin derivatives, benzoporphyrin monoacid derivatives (BPD-MA), monoacid ring “a” derivatives, tetracyanoethylene adducts of benzoporphyrin, dimethyl acetylenedicarboxylate adducts of benzoporphyrin, endogenous metabolic precursors, 6-aminolevulinic acid, benzonaphthoporphyrazines, naturally occurring porphyrins, ALA-induced protoporphyrin IX, synthetic dichlorins, bacteriochlorins of the tetra(hydroxyphenyl) porphyrin series, purpurins, tin and zinc derivatives of octaethylpurpurin, etiopurpurin, tin-etio-purpurin, porphycenes, chlorins, chlorin e6, mono-1-aspartyl derivative of chlorin e6, di-1-aspartyl derivative of chlorin e6, tin (IV) chlorin e₆, meta-tetrahydroxyphenylchlorin, chlorin e6 monoethylendiamine monamide, verdins such as, but not limited to zinc methylpyroverdin (ZNMPV), copro II verdin trimethyl ester (CVTME) and deuteroverdin methyl ester (DVME), pheophorbide derivatives, and pyropheophorbide compounds, texaphyrins with or without substituted lanthanides or metals, lutetium (III) texaphyrin, and gadolinium (III) texaphyrin.

In some embodiments, the photosensitizer of the present invention is selected among chlorins and bacteriochlorins that are porphyrin derivatives, however these have the unique property of hydrogenated exo-pyrrole double bonds on the porphyrin ring backbone, allowing for absorption at wavelengths greater than 650 nm. Chlorins are derived from chlorophyll, and modified chlorins such as meta-tetra hydroxyphenylchlorin (mTHPC) have functional groups to increase solubility. Bacteriochlorins are derived from photosynthetic bacteria and are further red-shifted to ^{˜}740 nmn. In particular, the photosensitizer of the present invention is meta-tetra hydroxyphenylchlorin (mTHPC) (or 5,10,15,20-Tetrakis(3-hydroxyphenyl)chlorin).

In some embodiments, the photosensitizer of the present invention is selected among purpurins, porphycenes, and verdins that are porphyrin derivatives that have efficacies similar to or exceeding hematoporphyrin. Purpurins contain the basic porphyrin macrocycle, but are red-shifted to ˜715 nm. Porphycenes have similar activation wavelengths to hematoporphyrin (635 nm), but have higher fluorescence quantum yields. Verdins contain a cyclohexanone ring fused to one of the pyrroles of the porphyrin ring. Phorbides and pheophorbides are derived from chlorophylls and have 20 times the effectiveness of hematoporphyrin. Texaphyrins are new metal-coordinating expanded porphyrins. The unique feature of texaphyrins is the presence of five, instead of four, coordinating nitrogens within the pyrrole rings. This allows for coordination of larger metal cations, such as trivalent lanthanides. Gadolinium and lutetium are used as the coordinating metals.

In some embodiments, the photosensitizer of the present invention is selected from the group consisting of cyanines, merocyanines, phthalocyanines with or without metal substituents, chloroaluminum phthalocyanine with or without varying substituents, sulfonated aluminum PC, ring-substituted cationic PC, sulfonated A1Pc, disulfonated and tetrasulfonated derivative, sulfonated aluminum naphthalocyanines, naphthalocyanines with or without metal substituents and with or without varying substituents, tetracyanoethylene adducts, nile blue, crystal violet, azure β chloride, rose bengal, benzophenothiazinium compounds, and phenothiazine derivatives including methylene blue. Cyanines are deep blue or purple compounds that are similar in structure to porphyrins. However, these dyes are much more stable to heat, light, and strong acids and bases than porphyrin molecules. Cyanines, phthalocyanines, and naphthalocyanines are chemically pure compounds that absorb light of longer wavelengths than hematoporphyrin derivatives with absorption maximum at about 680 μm. Phthalocyanines, belonging to a new generation of substances for PDT are chelated with a variety of metals, chiefly aluminum and zinc, while these diamagnetic metals enhance their phototoxicity. A ring substitution of the phthalocyanines with sulfonated groups will increase solubility and affect the cellular uptake. Less sulfonated compounds, which are more lipophilic, show the best membrane-penetrating properties and highest biological activity. The kinetics are much more rapid than those of hematoporphyrin derivative (HPD), with high tumor to tissue ratios (8:1) reached after 1-3 hours. The cyanines are eliminated rapidly.

Other photosensitizers of the invention include, but are not limited to, Diels-Alder adducts, dimethyl acetylene dicarboxylate adducts, anthracenediones, anthrapyrazoles, aminoanthraquinone, phenoxazine dyes, chalcogenapyrylium dyes such as cationic selena and tellurapyrylium derivatives, cationic imminium salts, tetracyclines and other photosensitizers that do not fall in either of the aforementioned categories have other uses besides PDT, but are also photoactive.

As used herein, the term “load” refers to the introduction or insertion of a substance or object into or onto a MSC-EV of the present invention. As used herein, the term “loading” refers to introducing or inserting a substance or object into or onto the MSC-EV of the invention.

In some embodiments, the MSC-EVs of the present invention are prepared by any method well known in the art. In some embodiments, the MSC-EVs of the present invention are prepared by methods for 3D culture that are well known in the art, and include, but are not limited to standard culture in 2D flasks, hanging drop culture, culturing on matrices, culturing on microcarriers, culturing on synthetic extracellular scaffolds, culturing on chitosan membranes, culturing under magnetic levitation, suspension culture in rotating bioreactors, or culturing under non-contact inhibition conditions. See, e.g., Haycock J W. (2011). “3D cell culture: a review of current approaches and techniques.”. Methods Mol Biol. 695: 1-15; Lee, J; Cuddihy M J, Kotov N A. (14 Mar. 2008). Three-dimensional cell culture matrices: state of the art. doi:10.1089/teb.2007.0150; Pampaloni, Francesco (October 2007). “The third dimension bridges the gap between cell culture and live tissue”. Nature Reviews 8: 839-845; and Souza, Glauco (14 Mar. 2010). “Three-dimensional tissue culture based on magnetic cell levitation”. Nature Nanotechnology: 291-296; the entire content of each are hereby incorporated by reference.

In some embodiments, the MSC-EVs of the present invention are prepared by the system culture described in WO2019/002608. In particular, the MSC-EVs of the present invention are prepared according to the method described in the EXAMPLE. More particularly, the method involves a fluid system comprising at least one container, a liquid medium contained by the container and producer cells, characterized in that it also comprises microcarriers suspended in the liquid medium, the majority of the producer cells being adherent to the surface of the microcarriers, and a liquid medium agitator, the agitator and the dimensions of the container being capable of controlling a turbulent flow of the liquid medium in the container.

Thus, a further object of the present invention thus relates to a method of preparing a MS-EV of the present invention comprising the steps consisting of i) causing a turbulent flow of a culture medium in a container, wherein the culture medium comprises the mesenchymal stem cells adhering to the surface of microcarriers, the microcarriers being in suspension in the culture medium and wherein the culture medium also comprises an amount of the photosensitizer, and then ii) collecting the produced extracellular vesicles from the liquid medium.

Typically, the microcarriers are microbeads. Commercially available media may be used for the growth, culture and maintenance of MSCs. Such media include but are not limited to Dulbecco's modified Eagle's medium (DMEM).

The MSC-EVs of the present invention is particular suitable for photodynamic therapy (PDT). For instance, the MSC-EVs of the present invention can be used in photodynamic therapy to inhibit the growth of, or kill a tumor cell. As used herein, the term “photodynamic Therapy” or “PDT” comprises administration of a photosensitizer followed by irradiation thereof, such that a reactive species is produced. The specificity of the photochemical reaction can be triggered by selecting the proper wavelength and specific photosensitizer to be used depending on the biologic effect desired.

Accordingly, a further object of the present invention relates to methods of reducing tumor cell growth and/or proliferation in a subject in need thereof comprising the steps of i) administering to the subject a therapeutically effective amount of a population of MSC-EVs of the present invention; ii) light-activating the photosensitizer loaded on the extracellular vesicles to produce cytotoxic species; and iii) thereby inhibiting the tumor cell growth and/or proliferation.

Typically, the photosensitizer can be activated at the target site with lasers or other light sources via optical fibres or any other appropriate method. The MSC-EVs of the present invention of the present invention must be photoactivated to induce their intended biological effect. For instance, the photoactivating light can be delivered to the target site from a conventional light source or from a laser. Target tissues are illuminated, usually with red light from a laser. Given that red and/or near infrared light best penetrates mammalian tissues, photosensitizers with strong absorbances in the approximately 600 nm to 900 nm range are optimal for PDT. Delivery can be direct, by transillumination, or by optical fiber. Optical fibers can be connected to flexible devices such as balloons equipped with light scattering medium. Flexible devices can include, for example, laparoscopes, arthroscopes and endoscopes.

Thus in some embodiments, the photodynamic therapy of the present invention is performed by photodynamic therapy by coelioscopy or laparoscopy or thoracoscopy or any solution for locoregional treatment in a serosa location as peritoneal cavity or pleural cavity.

In some embodiment, the photodynamic therapy of the present invention is performed by photodynamic therapy delivered by pressurized intraperitoneal aerosol chemotherapy (PIPAC) approach, similar PIPAC approach, PITAC (Pressurized Intrathoracic aerosol chemotherapy) or similar PITAC approach.

As used herein, the term “pressurized intraperitoneal aerosol chemotherapy (PIPAC)” has its general meaning in the art and refers to a new minimally invasive approach involving spraying drugs, i.e the population of MSC-EVs of the present invention, directly into the abdomen of a subject during a laparoscopy in the form of an aerosol. PIPAC approach are known to those of skill in the art and are briefly described in in several article [Graversen et al. Pleura Peritoneum. 2018; Nadiradze et al. Cancers (Basel). 2020; S. J. Tate et al. BJS Open. 2020; Alyami M et al. Lancet Oncol. 2019].

As used herein, the term “pressurized intrathoracic aerosol chemotherapy (PITAC)” has its general meaning in the art and refers to a new minimally invasive approach involving spraying drugs, i.e the population of MSC-EVs of the present invention, directly into the thoracic space of a subject during a thoracoscopy in the form of an aerosol. PITAC approach are known to those of skill in the art and are briefly described in in several article [Robella M et al. Anticancer Res, 2018; Khosrawipour V et al. Mol Clin Oncol. 2020; Taibi A et al. Surg Oncol. 2020].

In some embodiments, it may be desirable to administer population of MSC-EVs of the present invention locally to the area in need of treatment; this can be achieved, for example, by local infusion during surgery, by injection, by means of a catheter, or by means of an implant, said implant being of a porous, non-porous, or gelatinous material, including membranes, such as silastic membranes, or fibers.

In some embodiment, the population of MSC-EVs of the present invention can be administrated to the area in need of treatment as an aerosol.

In some embodiment, the population of MSC-EVs of the present invention can be administrated to the area in need of treatment as an aerosol by using PIPAC or PITAC approach, or similar approach.

Thus in some embodiment, the population of MSC-EVs of the present invention can be administrated to the area in need of treatment by spraying said the population of MSC-EVs of the present invention, directly into the area in need of a subject during a laparoscopy or a thorascoscpopy in the form of an aerosol.

Following administration of the population of MSC-EVs of the present invention, it is necessary to wait for the photosensitizer to reach an effective tissue concentration at the target site before photoactivation. The duration of the waiting step will vary, depending on factors such as route of administration, target location, and biodistribution the extracellular vesicles. The waiting period should also take into account the rate at which MSC-EVs of the present invention are degraded and thereby dequenched in the target tissue. Determining a useful range of waiting step duration is within ordinary skill in the art and may be optimized by utilizing fluorescence optical imaging techniques.

The population of MSC-EVs of the present invention is in particularly useful for the treatment of subject suffering from cancer occurring in body cavity.

As used herein, the term “cancer occurring in body cavity” (also known as serosal cancer location) refers to cancer which start in a body cavity. A body cavity is any space or compartment, or potential space in the body. Cavities is lined with a layer of cells and is filled with fluid, to protect the organs from damage as the organism moves around. Body cavities form during development, as solid masses of tissue fold inward on themselves, creating pockets in which the organs develop.

According to the invention, cancers occurring in body cavity include, but are not limited to, nasal cancer, oral cancer, mesothelioma, pleural metastasis bladder cancer, uterine cancer, pancreatic cancer, esophageal cancer, stomach cancer and peritoneal carcinomatosis.

In particularly, the population of MSC-EVs of the present invention is useful for the treatment of subject suffering from cancer selecting from the list consisting of: Head and neck cancer, mesothelioma, pleural metastasis, bladder cancer, uterine cancer, pancreatic cancer, esophageal cancer, stomach cancer and peritoneal carcinomatosis.

Thus a further object of the present invention relates to a method of treating cancer occurring in body cavity in a subject in need thereof, comprising the steps of i) administering to the subject a therapeutically effective amount of a population of MSC-EVs of the present invention; ii) light-activating the photosensitizer loaded on the extracellular vesicles to produce cytotoxic species; and iii) thereby inhibiting the tumor cell growth and/or proliferation.

In some embodiment, the population of MSC-EVs of the present invention can be administrated to the subject by performing PIPAC or PITAC approach.

In some embodiment, the population of MSC-EVs of the present invention can be administrated to the subject by spraying said the population of MSC-EVs of the present invention, directly into the abdomen or pleural cavity during a laparoscopy or a thoracoscopy in the form of an aerosol.

In some embodiments, steps i) and ii) may be repeated at least 2, 3, 4, 5, 6, 7, 8, 9, or 10 times.

As used herein, the term “head and neck cancer” has its general meaning in the art and refers to cancer in the larynx, throat, mouth, or nose. Head ans neck cancers usually begin in the squamous cells that line the moist, mucosal surfaces inside the head and neck (for example, inside the mouth, the nose, and the throat). These squamous cell cancers are often referred to as squamous cell carcinomas of the head and neck. Head and neck cancer include oral cavity cancer (also called mouth cancer), throat cancer such as laryngeal cancer or pharyngeal cancer, nasal and paranasal cancer.

As used herein, the term “mesothelioma” has its general meaning in the art and refers to cancer that develops from the thin layer of tissue that covers many of the internal organs (known as the mesothelium). Mesothelioma include pleural mesothelioma, peritoneal mesothelioma.

As used herein, the term “pleural metastasis” has its general meaning in the art and refers to cancer that has spread from another organ to the thin membrane (pleura) surrounding the lungs. Pleural metastases are generally associated with metastatic adenocarcinoma and are frequently associated with tumors of the lung, breast, pancreas, and stomach.

As used herein, the term “bladder cancer” has its general meaning in the art and refers to abnormal growth of the cells of the bladder.

As used herein, the term “uterine cancer”, also known as womb cancer, has its general meaning in the art and refers to cancer that develops from the tissues of the uterus. Uterine cancers include endometrial cancer and uterine sarcomas.

As used herein, the term “pancreatic cancer” has its general meaning in the art and refers to abnormal growth of cells in the pancreas. Pancreatic cancer includes exocrine pancreatic cancer, such as pancreatic ductal adenocarcinoma, and neuroendocrine pancreatic cancer.

As used herein, the term “stomach cancer”, also known as gastric cancer has its general meaning in the art and refers to an abnormal growth of cells that begins in the stomach. Stomach cancer include, gastrointestinal stromal tumor, gastrointestinal carcinoid tumor and gastric carcinoma such as gastric adenocarcinomas.

As used herein, the term “peritoneal carcinomatosis” refers to the neoplastic involvement of the peritoneum, typically seen as wide-spread seeding or growth of tumor masses or metastases. Peritoneal carcinomatosis can result from primary or secondary carcinomas. Primary peritoneal carcinomas arise from peritoneum cells and since the mesothelium of the peritoneum and the germinal epithelium of the ovary have the same embryologic origin, the peritoneum retains the multipotentiality allowing for the development of a primary carcinoma that can then spread within the peritoneal cavity. Primary carcinomas that cause peritoneal carcinomatosis and are contemplated for treatment using the disclosed methods and agents include malignant mesothelioma, benign papillary mesothelioma, desmoplastic small round cell tumors, peritoneal angiosarcoma, leiomyomatosis peritonealis disseminata (LPD), and peritoneal hemangiomatosis. Additionally, ovarian cancer arising in women after bilateral oophorectomy is included as a primary peritoneal cancer that can result in peritoneal catcinomatosis. Much more commonly, peritoneal carcinomatosis results from a cancer that arises in an anatonomically separate location and later metastasizes to the peritoneal cavity. Numerous cancers can produce peritoneal carcinomatosis including cancers of the endometrium, fallopian tubes, ovaries, uterus, colon, rectum, small bowel, gall bladder, bile duct, appendix, stomach, pancreas, liver and breast.

In some embodiments, the peritoneal carcinomatosis results from a colorectal cancer. As used herein, the term “colorectal cancer” includes the well-accepted medical definition that defines colorectal cancer as a medical condition characterized by cancer of cells of the intestinal tract below the small intestine (i.e., the large intestine (colon), including the cecum, ascending colon, transverse colon, descending colon, sigmoid colon, and rectum). Additionally, as used herein, the term “colorectal cancer” also further includes medical conditions, which are characterized by cancer of cells of the duodenum and small intestine (jejunum and ileum).

In some embodiments, the peritoneal carcinomatosis results from an ovarian cancer. As used herein, “ovarian cancer” or “ovarian tumor” includes any tumor, cell mass or micrometastasis derived from, or originating from cells of the ovary. This includes tumors originating from the epithelial cell layer (serous) of the ovary. Ovarian cancer further includes secondary cancers of ovarian origin and further includes recurrent or refractory disease.

In some embodiments, the peritoneal carcinomatosis is pseudomyxoma peritonei, the peritoneal dissemination of an appendiceal mucinous epithelial neoplasm, a relatively slow growing cancer that is characterized by the excessive production of mucinous ascites. (Smeenk R M, et al. Pseudomyxoma peritonei. Cancer Treat Rev 2007, 33:138-145).

The population of MSC-EVs of the present invention is in particularly useful for the treatment of subject suffering from peritoneal carcinomatosis or pleural metastasis,

Thus a further object of the present invention relates to a method of treating pleural metastasis in a subject in need thereof, comprising the steps of i) administering to the subject a therapeutically effective amount of a population of MSC-EVs of the present invention; ii) light-activating the photosensitizer loaded on the extracellular vesicles to produce cytotoxic species; and iii) thereby inhibiting the tumor cell growth and/or proliferation.

In some embodiment, the population of MSC-EVs of the present invention can be administrated to the subject by performing PITAC approach.

In some embodiment, the population of MSC-EVs of the present invention can be administrated to the subject by spraying said the population of MSC-EVs of the present invention, directly into the pleural cavity during a thoracoscopy in the form of an aerosol.

In some embodiments, steps i) and ii) may be repeated at least 2, 3, 4, 5, 6, 7, 8, 9, or 10 times.

Thus a further object of the present invention relates to a method of treating peritoneal carcinomatosis in a subject in need thereof, comprising the steps of i) administering to the subject a therapeutically effective amount of a population of MSC-EVs of the present invention; ii) light-activating the photosensitizer loaded on the extracellular vesicles to produce cytotoxic species; and iii) thereby inhibiting the tumor cell growth and/or proliferation.

In some embodiment, the population of MSC-EVs of the present invention can be administrated to the subject by performing PIPAC approach.

In some embodiment, the population of MSC-EVs of the present invention can be administrated to the subject by spraying said the population of MSC-EVs of the present invention, directly into the abdomen during a laparoscopy in the form of an aerosol.

In some embodiments, steps i) and ii) may be repeated at least 2, 3, 4, 5, 6, 7, 8, 9, or 10 times.

As used herein, the term “treatment” or “treat” refer to prophylactic, palliative or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. In some embodiment, the treatment may be administered to a subject whose tumor has been removed by surgery. In some embodiment, the treatment may be administered to a subject whose tumor has not been removed.

By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The population of MSC-EVs of the present invention can be administered in a pharmaceutically acceptable excipient, such as water, saline, aqueous dextrose or glycerol. The compositions can also contain other medicinal agents, pharmaceutical agents, adjuvants, carriers, and auxiliary substances such as wetting or emulsifying agents, and pH buffering agents. Standard texts, such as Remington: The Science and Practice of Pharmacy, 17th edition, Mack Publishing Company, incorporated herein by reference, can be consulted to prepare suitable compositions and formulations for administration, without undue experimentation. Suitable dosages can also be based upon the text and documents cited herein. A determination of the appropriate dosages is within the skill of one in the art given the parameters herein. Compositions of the present invention are administered in an appropriate way according to its composition. Available routes of administration include subcutaneous, intramuscular, intraperitoneal, intradermal, oral, intranasal, intrapulmonary (i.e., by aerosol), intravenously, intramuscularly, subcutaneously, intracavity, intrathecally or transdermally, alone or in combination with tumoricidal antibodies. Therapeutic compositions of MSC-EVs of the present invention are often administered by injection, mainly intraperitoneal or intravenous, or by gradual perfusion.

The invention will be further illustrated by the following figures and examples. However, these examples and figures should not be interpreted in any way as limiting the scope of the present invention.

### FIGURES

FIG. 1. Experimental set-up for mTHPC-EV production by turbulence.

A. Schematic picture of the setup of 3D culture cell and optical micrograph of microcarriers carrying MSC at confluence (A1) and one isolated imaged with a epiflurescence imaging microscope (A2-3-4, with respectively DAPI, mTHPC and the two channels). Size poydispersion was measured by NTA (B). Size distribution histograms obtained by nanoparticle tracking analysis for turbulence EVs. A single EV is illustrated with SEM (C), with a scale bar=100 nm.

FIG. 2. Biodistribution of mTHPC in colorectal and ovarian murine model of peritoneal carcinomatosis.

Tissue distribution of mTHPC after intraperitoneal injection at 4 h, 15 h, 24 h and 48 h depending on vectorization type. Results are presented as mTHPC mass concentration in each organ (ng mTHPC/mg tissue) (left) and mTHPC tumoral selectivity is highlighted by the ratio tumoral/tissue concentration (right) in colorectal carcinomatosis for (A) free mTHPC, (B) EVs-mTHPC, (C) Foslip® and (D) in ovarian carcinomatosis for EVs-mTHPC. (E) Direct comparison of mTHPC concentration at t=24 h for all the investigated organs with the 3 types of vectorization. (F) Direct comparison of mTHPC concentration in tumor, liver, kidneys and bowel at 4, 15, 24 and 48 h post-administration depending on mTHPC formulation: free mTHPC, Foslip®, EVs-mTHPC n=4 mice per time point and vectorization type; data are represented as mean±SEM (two-way ANOVA, * p=0.02, *** p<0.0001).

FIG. 3. Model of peritoneal carcinomatosis, experimental set-up and EVs-mTHPC dose determination.

Picture of (A) representative intraperitoneal disseminated nodules of CT26 in mouse abdomen and experimental set-up for PDT with laser beam focusing on the peritoneal cavity (B-C): mice underwent a laparotomy before illumination (λ=650 nm at 0.1 W/cm² for 100 sec). The peritoneum and the skin were separately sutured immediately after irradiation. The number of mice (without or with carcinomatosis) dead in the following 24 h post treatment is summarized in the table (D). The dose of 0.15 mg/kg (half of the lethal dose) was identified as suitable: mice responded positively to the treatment and histological analysis of nodules displays necrosis (E2) contrary to 0.05 mg/kg which induces no sign of necrosis (E1) (HE staining at 20× magnification). Liver injury with hepatocytes necrosis (area below dotted line, F) was observed after PDT at 0.30 mg/kg EVs-mTHPC but not at 0.15 mg/kg.

FIG. 4: Outcome of PDT treatments in colorectal PM as function of the PS formulation. (A) Necrosis value of tumor nodules evaluated through H&E histological analysis: the necrosis value (0-4) reflects the spatial extent of necrosis (n=20-25 nodules per control group and n=20 tumor nodules per PDT group, Kruskal Wallis test, *** p<0.0001). (B) Apoptosis evaluated using a TUNEL assay (n=6 per control group and n=10 per PDT group, Mann-Whitney non-parametric test, *p=0.04, **p=0.006) (C). Representative fluorescence microscopy of tissue cryosection stained with anti-CD31 antibody (scale bar=200 μm) (A: no treatment, B: laser, C: mTHPC, D: EVs-mTHPC, E: mTHPC+laser, F: EVs-mTHPC+laser) and (G) Quantification of CD31 fluorescence signal (n=15 per treatment, *p=0.03, **p=0.003, *** p<0.0001). (D). Quantification of Ki67 tumor proliferation index and immune cell infiltration in tumors: F4/80+macrophages, CD8+(E) and CD3+. T cells (n=30/group for KI67, F4/80 and CD3 analysis; and n=45/group for CD8 analysis, Kruskal Wallis test, *p=0.03, **p=0.003, *** p<0.0001).

FIG. 5. Representative image to demonstrate the volumetric analysis method with [18F]FDG TEP scan and global carcinomatosis evaluation using PCI.

A. A spherical volume of interest was drawn to encompass the whole hypermetabolic lesion in the FDG PET image. PET/CT fusion images were used to identify anatomical structures and to exclude colic activity. Representative photographs of tumors after second [18F]FDG TEP scan imaging. Nodes on parietal and diaphragmatic peritoneum was detected with an important sensitivity. B. Quantification of maximal standardized uptake value of peritoneal tumor and correlation between tumoral SUVmax after treatment/SUVmax before treatment. The development of the peritoneal carcinomatosis was slowed by PDT, n=5 mice per group, ** p=0.008. C. Peritoneal Carcinomatosis Index was significantly lower in PDT group, n=5 mice per group, *p=0.01.

FIG. 6. Kaplan-Meier survival curves for tumor-bearing mice and statistical comparisons of survival times after PDT.

A. Treatment groups (n=10 per group) included animals that received vectorized (or not) intraperitoneal photodynamic treatment (mTHPC 0.15 mg/kg, laser 10 J/cm2). Control groups (n=11 per group) included animals that received no treatment, laser illumination (10 J/cm2) without photosensitizer, mTHPC or EVs-mTHPC without laser illumination. B. Comparison between three PDT groups and no treatment group. C. A log rank test was carried out on survival data.

FIG. 7: Representative histological analysis of liver and kidneys 48 hrs after PDT.

Arrow indicate pathological changes in cell morphology (destruction of cell nucleus). Scale bars represent 120 μm for livers and 60 μm for kidneys.

### EXAMPLE

Methods

a/ Production of MSCm-EVs Encapsulating mTHPC Photosensitizer (EVs-mTHPC)

Cell Culture in Flasks

Murine mesenchymal stem cells (MSCm) were cultured in DMEM at 37° C. and 5% CO₂ in DMEM supplemented with 10% fetal bovine serum and 100 U/mL penicillin-steptomycin.

3D Cell Culture in Spinner Flask Bioreactors and EVs-mTHPC Production

MSCm were trypsinized, rinced with PBS, seeded in spinner flask bioreactor in DMEM complete medium containing 5 g of 200 μm Cytodex 1 dextran microcarriers (GE Healthcare); they were then submitted to 24 cycles of 45 min of rest interspersed with 3 minutes of gentle mixing at 30 RPM to ensure homogeneous adhesion of cells on microcarriers. After cell adhesion, continuous gentle mixing was performed until cell confluence on microcarriers (3-4 days). Meta-tetra(hydroxyphenyl)chlorin (mTHPC) (Biolitec, Germany) was added to the culture medium to obtain a final concentration of 100 μM for an overnight labelling. Before EV production was launched, complete medium+mTHPC was rinsed with 5 washing steps (with serum-free white DMEM medium, 100 U/mL penicillin-steptomycin) were performed to remove the non-encapsulated drug. Spinner flasks were then submitted to mixing at 144 RPM during 4 hours for EVs-mTHPC production.

EVs-mTHPC Isolation and Purification

After EV production, EVs-mTHPC were isolated from the conditioned culture medium. First, microcarriers and cell debris were eliminated by 2,000 g centrifugation for 10 min. The following ultracentrifugation at 100,000 g for 70 minutes allowed isolating the EVs-mTHPC, which were then resuspended in phosphate-buffered saline.

EVs-mTHPC Characterization (Size, Yield and mTHPC Quantification)

EV size distribution and concentration were determined by Nanoparticle Tracking Analysis (NTA) using a Nanosight $NS300 HS with a 405 nm laser. Before measurements, EVs were diluted to an appropriate concentration (between 3×10⁸ and 2×10⁹) with sterile PBS (confirmed to be particle-free by NTA measurement). For each sample, 5 movies of 30 s were recorded using a camera level of 16. Data were analyzed with NTA Analytical Software. The concentration of m-THPC in samples of purified EVs was determined by fluorescence spectroscopy. An EnSpire (Perkin Elmer) plate reader spectrometer was used at 410 nm excitation wavelength. The drug concentration was obtained from fluorescence emission at 655 nm based on a standard calibration curve of m-THPC. For m-THPC quantification in EVs, Triton X-100 was added at 0.3% final concentration in order to lyse EVs.

b/ Cell Culture

Mouse colon (CT26) and ovarian (ID8) cancer cell line genetically modified to stably express luciferase (CT26-Luc and ID-8) were used. The CT26-Luc cells were cultured in Dulbecco's Modified Eagle's Medium (DMEM) with L-Glutamine supplemented with 10% fetal bovine serum, 1% penicillin-streptomycin at 37° C. in 5% CO2 humidified atmosphere. The ID8-Luc cells were cultured in Dulbecco's Modified Eagle's Medium (DMEM) supplemented with 4% fetal bovine serum, 1% penicillin-streptomycin, 1% d′ITS, 1% de L-glutamine and 2.5 μg/mL of puromycin.

c/ In Vitro Experiments: Exposition to EVs-mTHPC and Laser Irradiation

The CT26-Luc and ID8-Luc cells were seeded in 24-well plates and incubated overnight. After PBS rinsing, the cells were incubated with XX μL of EVs-mTHPC at XX or XX μM during 24 h in the dark. Wells were then washed with PBS, covered with white DMEM and irradiated individually using a 650 nm diode laser featuring a fiber delivery system. The optical fiber was fixed so that the laser spot covers precisely the surface of the well (1.9 cm²). Cells were exposed to at a light fluence of 10 J/cm² (100 mW/cm² for 100 s). Cells were incubated for 24 h before cytotoxic assessment by the Alamar Blue test (Invitrogen), according to the supplier's instructions.

d/ Scanning Electron Micrograph (SEM) Observation

SEM was first used to visualize extracellular vesicles (EVs) at CT26 and ID8 tumoral cell surface. Tumoral cells were cultured in 8 well-chambers removable (Ibidi, 80841) for one day. Cells were washed with PBS and we replace with complete medium only, or containing EVs-mTHPC. After 24 hours, two well-chambers were illuminated at 10 J/cm². After 24 hours, cells were fixed 30 minutes with 4% paraformaldehyde and then further rinsed three times with PBS buffer. Dehydration was performed by rinsing the samples through graded ethanol/water mixtures (50%, 70%, 80%, 90%, and finally 100%, each step for 10 min at 4° C.).

e/ Animal Model

All animal experiments were performed in agreement with institutional animal use and care after approval by the local Ethics Committee (registration number for the carcinomatosis model experiment: APAFIS-8617). Six-week-old female BALB/c (provided by Charles River, Arbresle, France and weighing 18 g) and C57BL/6 mice were provided with food and water ad libidum, for colon and ovarian model respectively. The animals were allowed to acclimate to the facility for at least one week before being used for experiments. Colorectal model was induced with an intraperitoneal injection of 5·10⁴ CT26 cells resuspended in a volume of 200 μL of physiological saline per inoculum. Ovarian model was induced with an intraperitoneal injection of 10⁶ ID8-LUC cells suspended in 1 mL of medium. Between the days of IP injection and sacrifice, the wellbeing of the mice was checked twice a week through the search of any sign of pain, dehydration, changing in behavior or loss of weight.

f/ In Vivo Photodynamic Therapy (PDT)

The mTHPC and EVs-mTHPC were injected intraperitoneally with 200 μL of free mTHPC (in a solution of ethanol/polyethylene glycol 400/water at a 2/3/5 volume ratio) or vectorized mTHPC with EVs or liposome (Foslip®), at 0.15 mg/kg drug concentration. mTHPC [3,30,300,3000-(2,3-dihydroporphyrin-5,10,15,20-tetrayl)-tetraphenol] and its liposomal formulation (Foslip®) were kindly provided by Biolitec research GmbH (Jena, Germany). After 24 h, animals from both groups were anesthetized with isoflurane, a midline laparotomy was performed to allow invasive laser irradiation, and were then irradiated using a 650 nm laser at a fluence of 10 J/cm² (100 mW/cm² for 100 s). Two irradiations spots were successively realized to allow an illumination of the entire cavity and drops of saline were used to keep the tissue moist. A heating device was used to keep body temperature stable throughout the treatment until the animal was woken up. The peritoneum and the skin were separately sutured immediately after irradiation.

g/ mTHPC Biodistribution: Drug Extraction and Spectroscopy Measurements

Drug extraction mTHPC levels in organs after IP injection of mTHPC, EVs-mTHPC or Foslip®, were quantified using chloroform extraction based on a previous protocol described in Foster et al. (Foster et al, Translational Oncology (2010) 3, 135-141). Briefly, tumor-bearing animals (with colorectal carcinomatosis) were injected at 0.5 mg/kg mTHPC intraperitoneally. Either at 4 h, 15 h, 24 h or 48 h mice were sacrificed to determine mTHPC accumulation in different organs: tumors, liver, spleen, kidney, intestine, peritoneum, skin and lung. First, organs were weighed and grinded in PBS (1 mL per 10 mg of organ). The amount of 0.8 mL of MeOH per 10 mg of organ was added to the solution and vortexed; then, 0.8 mL of chloroform was added. The solution was centrifuged at 1400 rpm for 10 minutes to allow the separation of the aqueous and chloroform phases. The bottom layer consisting of the chloroform and the solubilized mTHPC was transferred for spectroscopy measurements. Fluorescence emission spectra were obtained by a fluorescence spectrophotometer using 415 nm excitation wavelength. Biodistribution of EVs-mTHPC was compared in ovarian carcinomatosis.

Ascitis smears were performed as soon as the sacrifice at 24 h, in ovarian model. Ten minutes after 4% paraformaldehyde fixation, cells were permeabilized with 0.1% Triton for 5 minutes and then blocked with 1% BSA for one hour. The cells were then incubated overnight at +4° C. with anti-firefly Luciferase antibody [EPR17789] (AlexaFluor 488) (ref ab237251) at 1/100 dilution. Tumoral cells presented in ascitis were detected with immunofluorescence analysis with a confocal microscope (Carl Zeiss Microscopy GmbH LSM 800). Colocalisation between tumoral cells expressing LUC and mTHPC fluorescence was observed (excitation 405 nm, emission 635-700 nm).

h/ Safety, Tolerability and Preliminary Anti-Tumor Activity

The maximum tolerated dose of EVs-mTHPC for intraperitoneal PDT was evaluated. Mice with (n=10) and without colorectal peritoneal metastasis (n=9) received three concentrations of mTHPC: 0.05 mg/kg, 0.15 mg/kg and 0.3 mg/kg. Drug injection was performed 8 days after IP inoculation of CT26 LUC cells. PDT was performed the day after IP injection of EVs-mTHPC; a midline xiphoid-pubic laparotomy was made to allows intraperitoneal laser illumination. Mice were sacrificed three days after PDT. Tumoral metastasis, liver and kidney were preserved in 4% paraformaldehyde for histological analysis.

i/ Effectiveness and Toxicity of PDT with EVs-mTHPC in Mouse Model of Colorectal Peritoneal Carcinomatosis

Effectiveness evaluation of PDT with EVs-mTHPC was performed with two experimentations, to assess early and long-term effectiveness. Treatment injection was made 12 days after IP inoculation of CT26, followed by invasive illumination 24 hours after. Six groups were performed for each experimentation: (i) no treatment, (ii) laser, (iii) free mTHPC, (iv) EVs-mTHPC, (v) free mTHPC+laser, (vi) EVs-mTHPC+laser.

Early effectiveness was evaluated 48 hours after illumination to compare: tumoral necrosis, apoptosis, macrophages invasion, lymphocyte recruitment, vascular damage and proliferative index result according to treatment. Long-term effectiveness consisted on a survival analysis function of the treatment.

Lesion Characterization by Pathology

Tumor were fixed in 4% paraformaldehyde, embedded in paraffin and sectioned at 4 μm to histological processing using hematoxilin/eosin (HE) and immunohistochemical stains. Depth of necrosis was blindly determined for each slide by a confirmed pathologist. Necrosis was evaluated using a semi-quantitative histology. Necrosis Value (NV) was determined according to the depth of the necrosis for each tumor nodule. The NV was correlated to the percentage of necrosis: 0: no necrosis, <25%=1; 25-50%=2; 50-75%=3; >75%=4. This technique was used to evaluate toxicity on live and kidney too.

For immunohistochemical detection of the chosen markers for tumor tissue, sections were deparaffined and subject to antigen retrieval methods validated for each of the primary antibodies. Sections were incubated overnight at 4° C. with primary antibodies (anti CD8 antibody (Cell signaling technology #98941), anti CD3 antibody (ab5690), anti F4/80 antibody (Cell signaling technology, #70076) and anti-Ki-67 antibody (ab16667), then incubated with secondary antibody and developed using the avidin-biotin complex method with 3,3′diaminobenzidine as chromogen. Histology and immunostaining preparations were performed on the Cochin HistIM Facility, Paris.

Antibody expression for each analysis was evaluated by the mean number of positive cells in 3 randomly chosen areas (×40 magnification) for 10-15 nodes by treatment. Quantitative analysis of immunostaining preparation was performed using the color segmentation ImageJ plugin developed by the Biomedical Imaging Group at the EPFL, Switzerland.

Immunofluorescent Analysis of Tumor

TUNEL assay: Cell apoptosis in tumour was detected by a FragEL DNA fragmentation (TUNEL) detection kit (Sigma, Roche, ref 11684795910). In brief the cryostat section were permeabilized with 0.1% Triton and 0.1% (citrate de sodium) in 10×PBS. The slides were then labeled with a TdT reaction mixture for 60 min and were mounted with a mounting solution containing 4′, 6-diamidino-2-phenylindole (DAPI). The apoptotic cells (green) and cell nucleus (blue) was examined using a fluorescence microscopy. The percentage of apoptotic cells was assessed in 2 randomly fields at 40× magnification. The apoptotic index was calculated with image J.

For image-based quantitative analysis of blood vessels, immunofluorescence detection of CD31 was made after one hour incubation with antiCD31 antibody (BD 553370,1/50) and 45 min incubation with second antibody (Alexa Fluor® 488, ThermoFisher, A11006, dilution 1/200)

Survival Analysis

Another 10 or 11 mice were selected from each group to evaluate the overall survival until 30 days. The end point was defined as mice death or the cachexia with the loss of 20% of the total weight that required the killing of the animal (as mentioned in the registration).

j/ Metabolic Activity Response of Peritoneal Metastases Measured by [18F]FDG PET/CT Scan after PDT with EVs-mTHPC

Ten days after CT26 IP injection, five mice were IP injected with EVs-mTHPC with IP illumination 24 h after. [18F]FDG PET/CT scan imaging were done before and 48 hours after PDT. Metabolic activity of various metastatic lesions was measured. Maximal standardized uptake value (SUVmax) was quantitatively used to determine 18F-FDG avidity. SUV was defined as the concentration of 18F-FDG divided by the injected dose, corrected for the weight of the mice and radioactive decay at scanning time [SUV=activity concentration/(injected dose/mice weight)]. The results were compared with five control mice, without treatment. We correlate this result with clinical extension of metastasis by calculating peritoneal carcinomatosis index (PCI) score.

k/ Statistics

All data were analyzed with the GraphPad Prism® version 7 software. Results were represented mean±SEM. comparison was performed using either a Mann-Whitney non-parametric test, or a Kruskal-Wallis test depending on the number of groups to compare. The survival curves were calculated by the Kaplan-Meier method, and the statistical significance of differences in the cumulative survival curves between the groups was evaluated by logrank test. The P values less than 0.05 were considered significant.

Results

Photosensitizer Synthesis: EVs-mTHPC

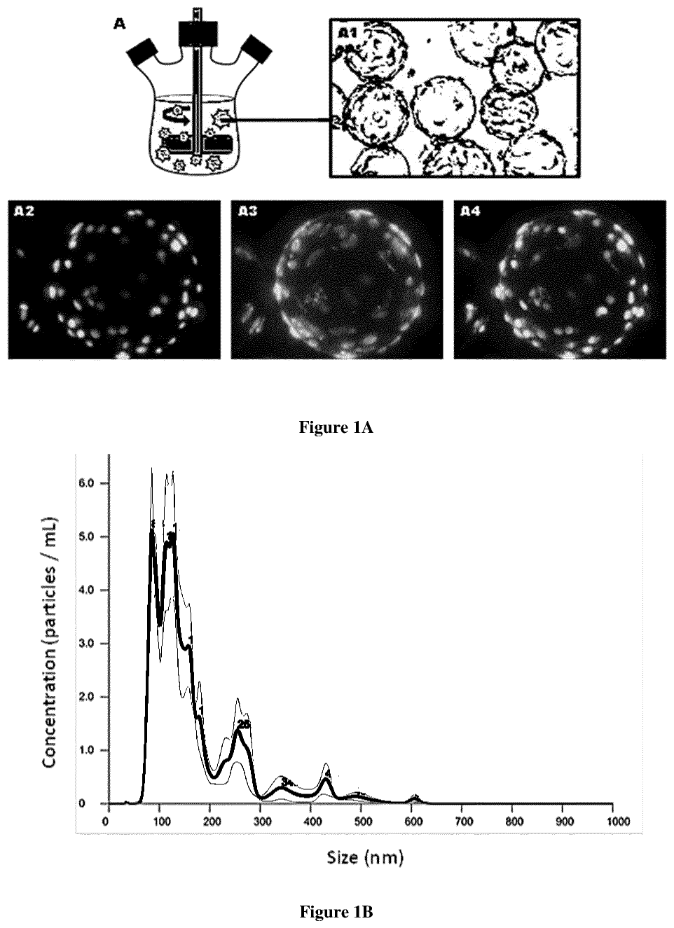

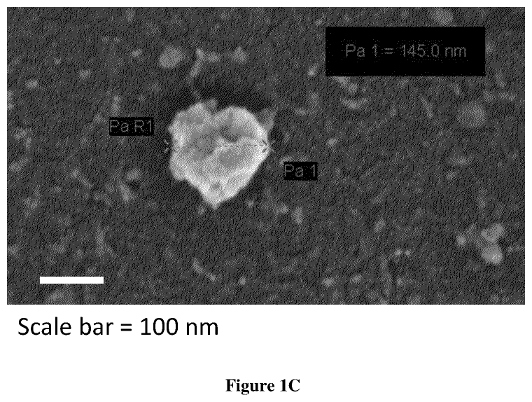

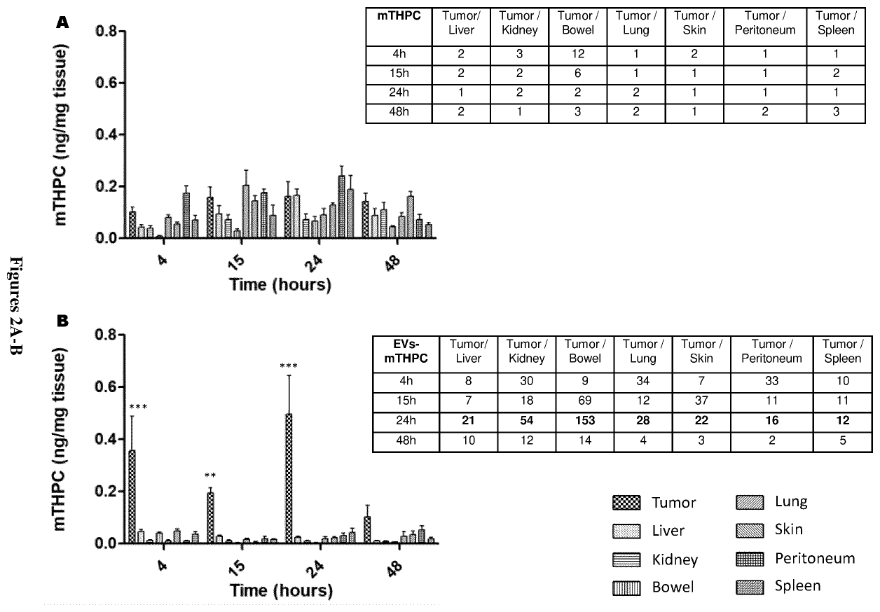

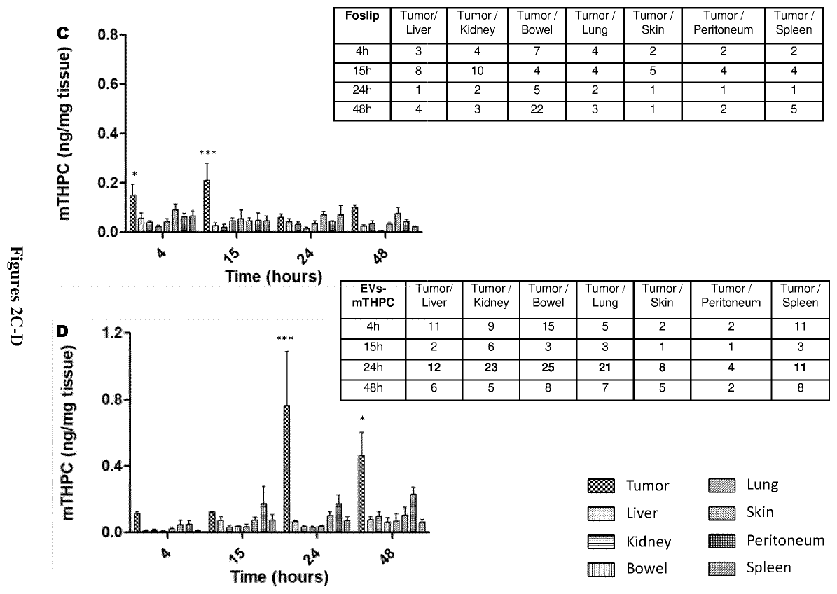

After reaching confluence on beads (as exemplified in FIG. 1.A), MSCs were incubated overnight with mTHPC. FIG. 1.A.2-4 displays MSCs on a bead after mTHPC labeling and evidences the cytoplasmic localization of the internalized mTHPC. With an initial number of cells of 32·10⁶ seeded on beads and after 3 days of cell division, the total quantity of EVs produced after mixing at 144 RPM during 4 hours was measured by NTA at 1.4×10¹³ EVs. Size distribution of EVs was analyzed both by NTA and SEM (FIG. 1.B-C): classical shape and typical polydisperse EV size range was observed, with a mean size of 175.2 nm±14.5. Diluted EV sample at 1.2×1012 EV/mL featured a concentration of 100 μM of mTHPC. To ensure EV stability throughout the duration of the study, EVs were stored at −80° C. No changes of EV shape nor mTHPC concentration was noticed.

In Vivo Biodistribution Distribution of mTHPC Function of Formulation

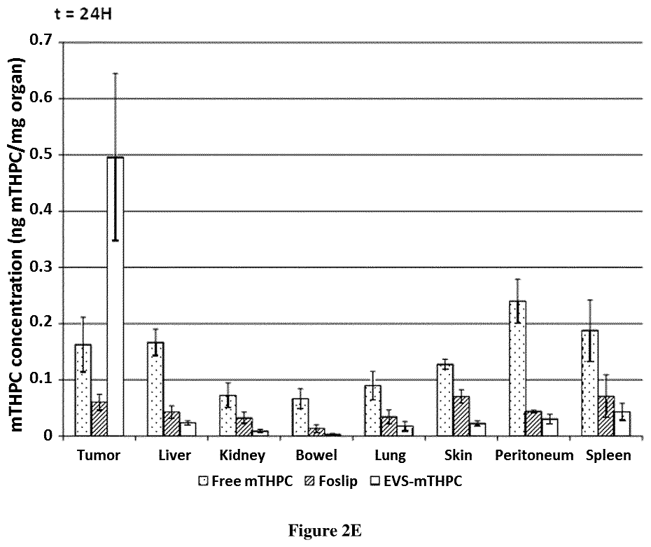

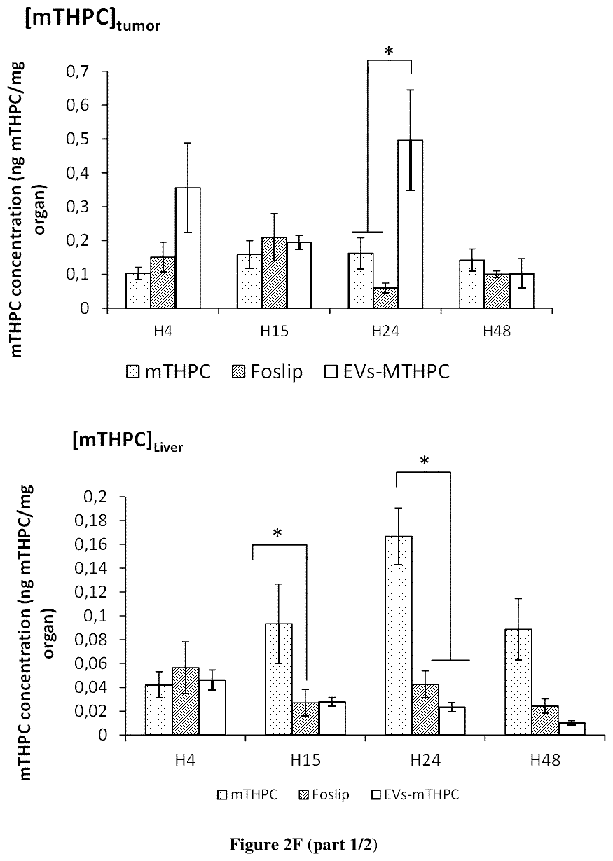

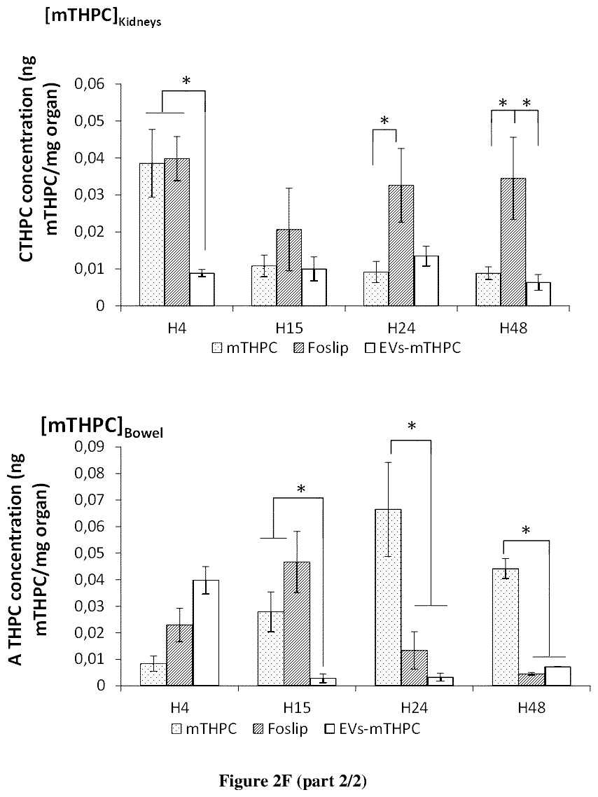

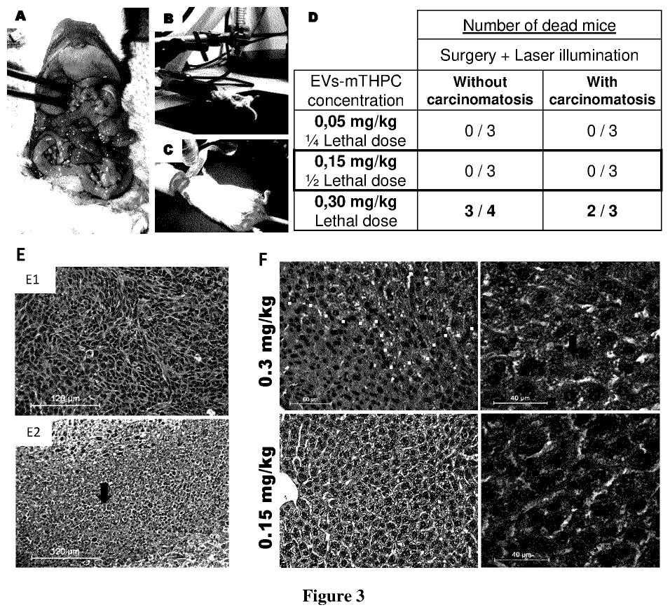

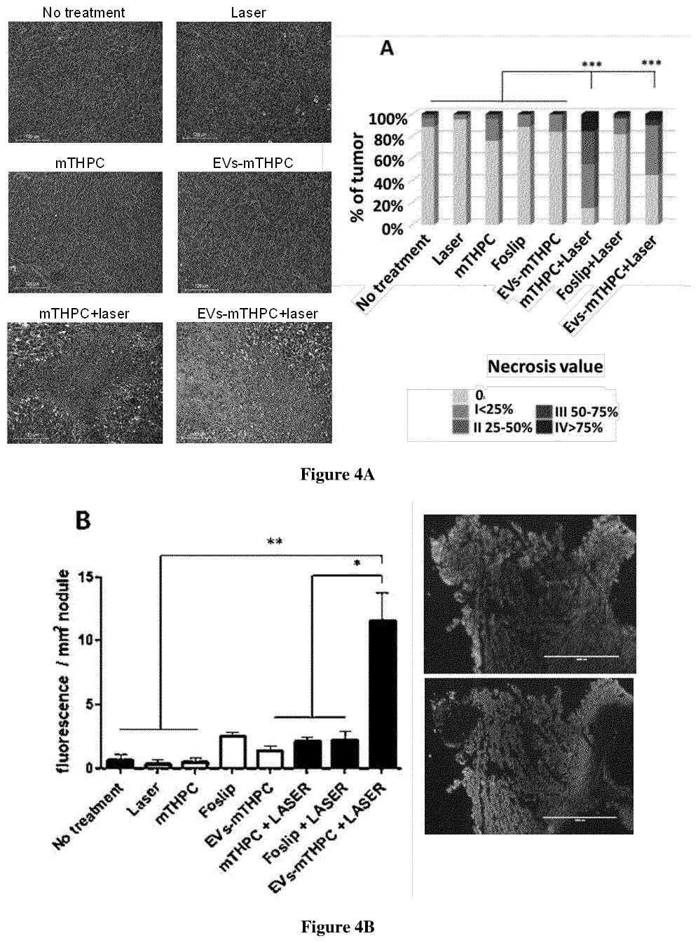

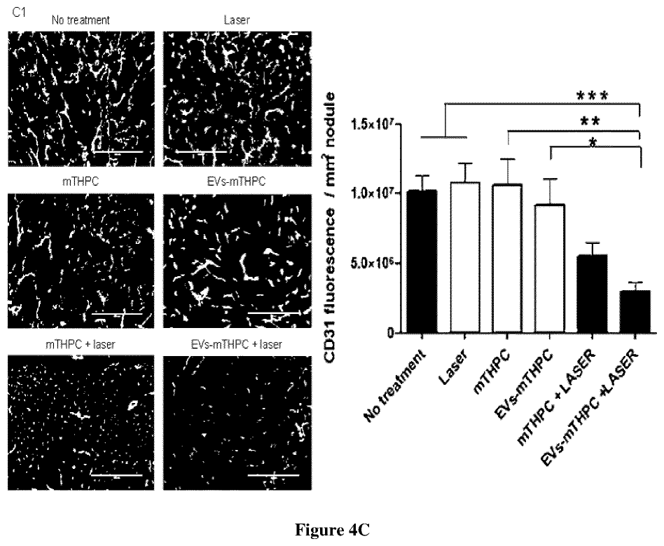

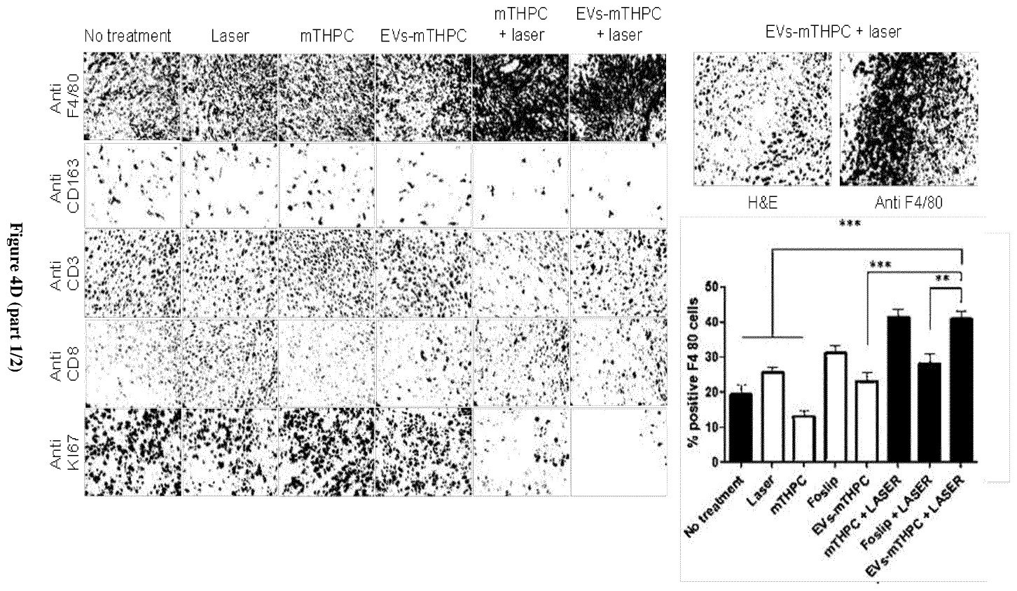

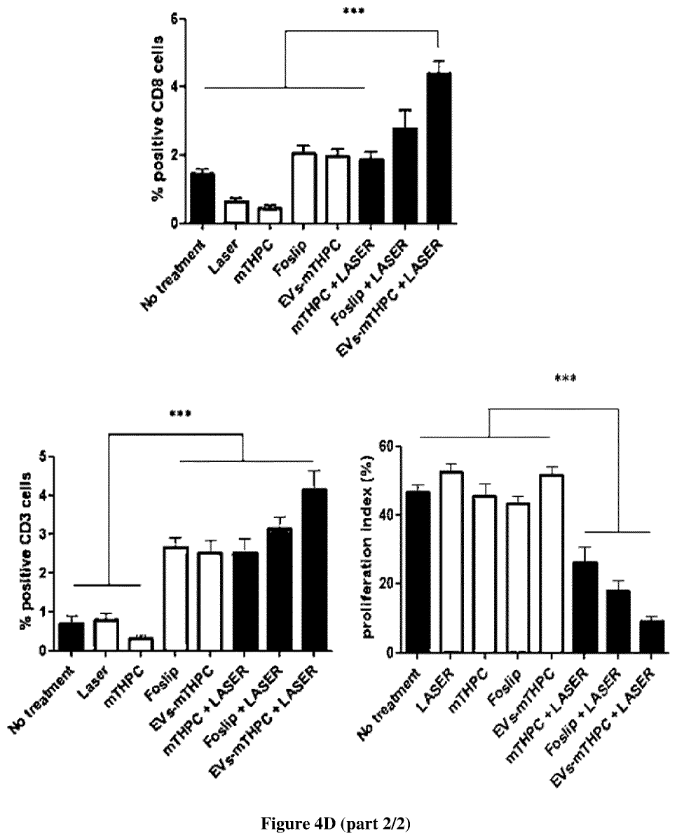

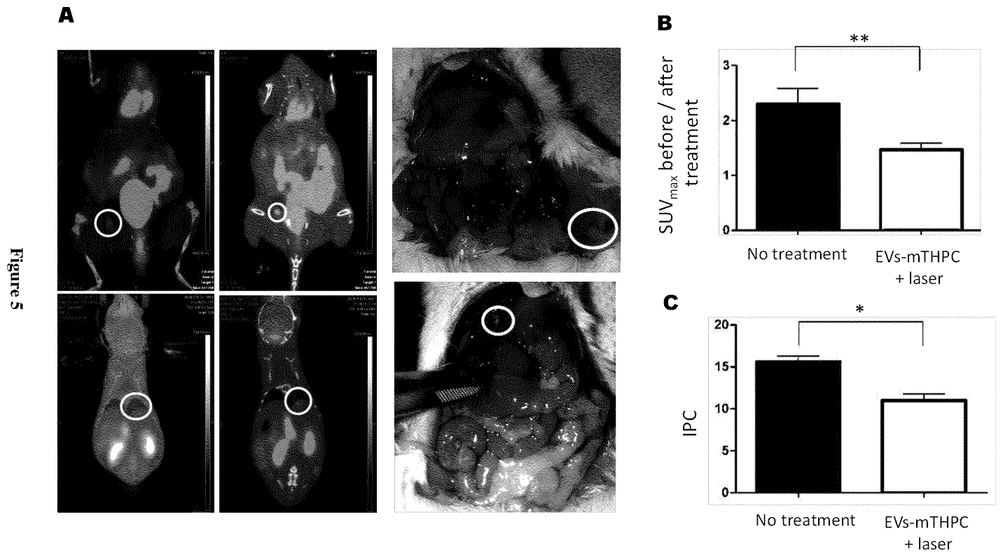

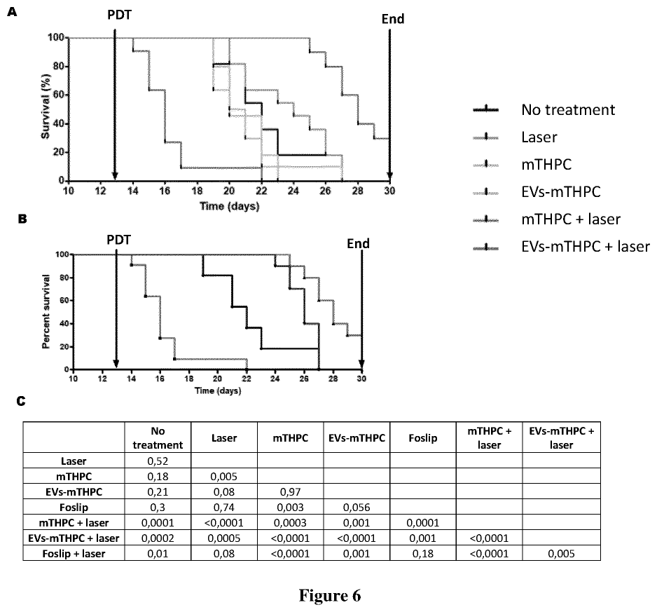

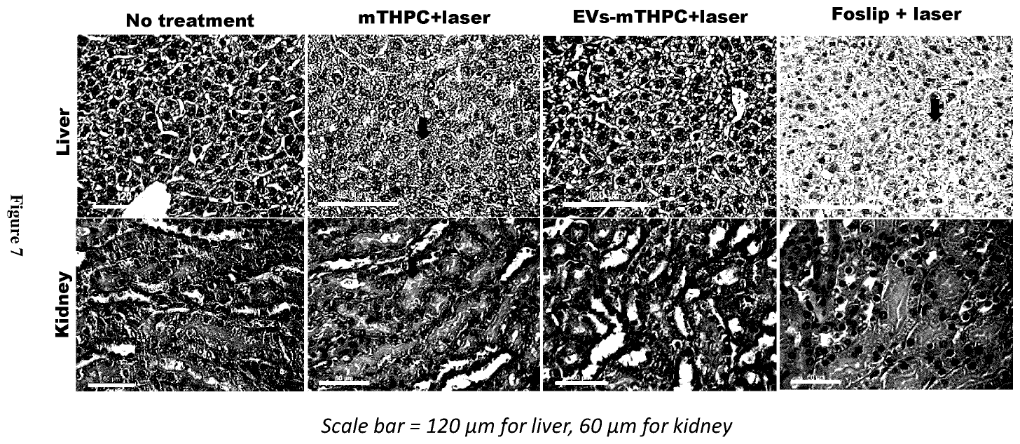

The mTHPC concentration as a function of time after injection is shown in FIG. 2. For all organs except the tumors, the error bars indicate the standard errors arising due to inter-animal variations. Whereas for tumor tissue, the error bars also partly reflect intra-animal differences.

In colorectal model of carcinomatosis, for tumor tissue, the mTHPC levels at 4 h and 24 h were higher for EVs nanovectorization than free or liposomal vectorization of mTHPC (4 h: 0.36±0.1 vs 0.10±0.01 or 0.15±0.04, and 24 h: 0.50±0.1 vs 0.16±0.05 or 0.06±0.01 ng/mg tissue respectively). At 15 h intra tumoral concentration were almost equivalent in three groups (0.19±0.02 vs 0.15±0.04 or 0.21±0.07 respectively) and at 48 h, we noted an elimination of mTHPC (0.10±0.04 vs 0.10±0.00 or 0.14±0.03 respectively). We observed for free mTHPC, the tumor-to-organ ratio did not change significantly with time and displayed a total average of 2.5. The average selectivity of mTHPC in tumor compared to other organs investigated is listed for the time points investigated, function of the mTHPC formulation. At 24 h, the tumor-to-organ ratio averaged was between 1 and 2 for free mTHPC and between 1 and 5 for liposomal formulation. The highest selectivity achieved with EVs formulation, with a ratio between 12 and 153 at 24 h. The biological nanovectorization of mTHPC allowed a 40 and 51 times higher selectivity in tumor-tissue compared to liposomal vectorization and free formulation respectively.

We analysed the EVs nanovectorization in a second model of carcinomatosis, from ovarian origin. The highest tumoral selectivity achieved at 24 h too, with a mean tumor-to-organ ratio at 15.

Safety, Tolerability and Preliminary Anti-Tumor Activity

The toxicity data for non-tumor bearing and tumoral mice treated with increasing doses of EVs-mTHPC is shown in FIG. 3. The majority of mice receiving an injection of 0.3 mg/kg EVs-mTHPC died less than 24 h after illumination: 75% (3 out of 4) of tumoral mice and 66% (2 out of 3) of non-tumoral mice. All mice injected with 0.05 and 0.15 mg/kg of mTHPC survived. Histological analysis of the liver indicated some histopathological changes after 0.30 mg/kg IP injection of EVs-mTHPC, with nuclear degradation and infiltration by inflammatory cells. The liver toxicity could explain death of mice. Tumoral histological analysis showed necrosis at 0.15 mg/kg, but no necrosis was detected at 0.05 mg/kg. 0.15 mg/kg appeared like the ½ lethal dose, with a preliminary antitumoral effectiveness.

Effectiveness of PDT in a Mice Model of Peritoneal Carcinomatosis from Colorectal Origin

A total of 94 tumors from control mice (n=27 untreated, n=23 laser, n=25 mTHPC, n=19 EVs-mTHPC) and 40 tumors from mice treated with PDT (n=20 per group) were analyzed by H&E staining (FIG. 3). PDT performed on carcinomatosis lesions at 650 nm with an irradiance 0.1 W/cm² for 100 sec induced necrosis in 72.5% of cases versus 13% with control treatment (mean NV control=0±0.06, mean NV mTHPC+laser=1.6±0.3, mean NV EVs-mTHPC+laser=1±0.2), p<0001. Intra tumoral necrosis was not extensive but involved both the center and periphery of the node. To further investigate the mode of death in the observed tissue damage areas, we used TUNEL staining to assay for apoptosis revealing that tumors after EVs-mTHPC+laser had a higher level of green fluorescence than others treatment. The mean fluorescence per mm² of tumor was significantly (p<0.05) higher after treatment with EVs-mTHPC+laser than in the other treatment groups: no treatment: 0.55±0.4, Laser: 0.54±0.8, mTHPC: 0.86±0.4, EVs-mTHPC: 1.42±0.4, mTHPC+laser: 2.17±0.2 versus EVs-mTHPC+laser: 11.60±2.2. We then compared the short-term outcome of PDT treatment on the colic PM 48 h after the single laser exposure for the three PS formulations. None of the formulations without laser irradiation induced tumor necrosis (dark toxicity) (FIG. 4A). Significant increases in necrosis values (NV) were observed in irradiated mice injected with the free drug (mTHPC+laser, mean NV=1.6±0.3) and with EVs-mTHPC (EVs-mTHPC+laser, NV=1±0.2) compared to control non exposed groups. Intra-tumor necrosis did not extend to the whole tumor volume but involved both the center and the periphery of the node (data not shown). Apoptosis level assessed by TUNEL was 5-fold higher in the laser-exposed group receiving EVs-mTHPC in comparison to the Foslip® and free drug (FIGS. 4B and 4D) and 36-fold higher than in control non-exposed groups. Proliferation index was reduced by a factor of 5 with photoactivated EVs-mTHPC versus 2 for the free drug and 3 for the Foslip® formulation.

To investigate the indirect anti-tumor effects of PDT on intraperitoneal dissemination, additional immunohistochemistry explored macrophages infiltration and lymphocyte recruitment. Macrophage intra-tumor invasion (anti F480 antibody) was significantly higher in mice treated with PDT (mTHPC+laser 41.45%±2.2, EVs-mTHPC+laser 41.04%±2.1) than control mice (untreated 19.34%±2.6; 25.78%±1.5, mTHPC 13.19%±1.4, EVs-mTHPC 23.26%±2.5), p<0.0001. Infiltrated inflammatory cells were seen in the necrosis zone among the PDT groups. The PM microenvironment was differently modified by the different treatments, particularly the immune cell tumor infiltrate. Noteworthy the tumor invasion of F4/80 macrophages (FIG. 4D) was significantly higher in mice treated with free drug or EVs-mTHPC and irradiated in comparison to the non-irradiated or laser only groups, but not in mice treated with Foslip® and irradiated. This result is in line with necrosis values that were maximal for PDT with free drug and EVs-mTHPC and could elicit tumor inflammation

The T-lymphocyte infiltration, unlike macrophage infiltration, was increased first by the EVs, and second by the PDT. Anti CD3 labeling in PDT treated mice after IP injection of EVs-mTHPC (4.14%±0.5) was twice as high than EVs-mTHPC (2.55%±0.3) and PDT group after free mTHPC IP injection (2.51%±0.3), in contrast with other control groups (untreated groups 0.71%±0.1; laser: 0.79%±0.1, mTHPC: 0.33%±0.07), p<0.0001. To explore defining T-cell category, we performed CD8 immunodetection, with the same results. CD8 detection in PDT treated mice after IP injection of EVs-mTHPC (4.40%±0.3) was twice as high than EVs-mTHPC (1.96%±0.2) and the PDT group after free mTHPC IP injection (1.88%±0.2), in contrast with other control groups (untreated groups 1.45%±0.13; laser 0.65%±0.08; mTHPC 0.44%±0.07), p<0.0001. Indeed, the number of CD3+ T cell markedly increased in tumors when both Foslip® or EVs-mTHPC were injected in comparison with the free drug and controls (FIG. 4D). However, laser irradiation amplified T-cell infiltration with the most prominent effect with EVs-mTHPC (FIG. 4D). The accumulation of effector CD8+ cell in tumors show a similar trend with increased recruitment in mice treated with EVs-mTHPC+laser (FIG. 4D). As the percentage of CD3+ and CD8+ within the nodules were equivalent, lymphocyte infiltration can be mainly composed of cytotoxic lymphocytes. Overall, PDT in colon PM induces a pro-inflammatory immune environment with inflammatory macrophages and cytotoxic T cell infiltration that is mostly promoted by mTHPC vectorization with MSC-derived EVs. Finally, a reduced expression of CD31 endothelial cell marker (FIG. 4C) after PDT, indicating vascular damages, with the most prominent effect in mice treated with EVs-mTHPC+laser. The percentage of CD3 and CD8 positive within the nodules being equivalent, lymphocyte infiltration was mainly composed of cytotoxic lymphocytes. Moreover, the lymphocyte infiltration increasing with EVs, probably mesenchymal nature of these vesicles permitted an immunomodulatory character.

Immunofluorescence staining further demonstrated that PDT treated tumors exhibited smaller CD31 (an endothelial cell marker) expression (mTHPC+laser: 0.55·10⁷±1.0·10⁶ and EVs-mTHPC+laser: 0.29·10⁷±0.6·10⁶) than control groups (no treatment 1.02·10⁷±1.0·10⁶, Laser: 1.09·10⁷±1.4·10⁶, mTHPC: 1.06·10⁷±1.4·10⁶, EVs-mTHPC: 0.92·10⁷±1.9·10⁶).

In total, PDT with mTHPC permitted an antitumoral action by necrosis, macrophages infiltration and vascular damage. mTHPC nanovectorization with EVs permitted a more cytotoxic PDT effect with apoptosis and lymphocyte recruitment.

In consequences, to further characterize the effect of PDT in peritoneal disseminated tumors proliferation, we performed immunohistochemical staining for Ki67. The mean Ki67-index was significantly lower in the PDT-treated group after injection of EVs-mTHPC (9.2%±1.4) compared to controls (no treatment 46.6%±2.2; Laser 52.7%±2.5; mTHPC 45.52±3.4; EVs-mTHPC 51.9%±2.3) and the group treated with PDT after free mTHPC injection (26.2%±4.5), p<0.0001.

Metabolic Activity Response of Peritoneal Metastases Measured by [18F]FDG PET/CT Scan after PDT with EVs-mTHPC

9 mice received the 2 PET/CT scan images: 5 untreated and 4 treated. The injection of [18]FDG could not be performed correctly in one treated mouse, with a diffusion of the product at the level of the tail. The mean SUVmax ratio 2^{nd}/1^{st} PET/CT scan imaging was significantly higher in the control group compared to the treated group (1.94±0.1 vs 1.45±0.06, p=0.008 respectively). The SUVmax of the 2^{nd} imaging was systematically higher than the 1^{st} imaging, with apparition of tumors on the 2^{nd} imaging. It shows the aggressiveness of this tumor model with exponential growth. PDT treatment did not block tumor development in our model but induced a slowdown. This treatment effectiveness was also expressed by the evaluation of PCI. Mean PCI was significantly higher in untreated mice compared to PDT-treated mice (16±0.8 vs 11±0.7 respectively, p=0.01). In addition, we showed that this imaging was very sensitive for infra-millimeter nodule in parietal peritoneal node (FIG. 5). However, PET/CT scan was not suitable for evaluating mesenteric tumor lesions in our model due to the intense hypermetabolism digestive tract despite the fasting period.

EVs-Nanovectorized PDT Leads to Significant Survival Advantage

At the end of the study, the survival rate of the EVs-mTHPC+laser group was 30%, with a median survival of 28 days, while there were no survivor in the other groups (median survival: 16, 20, 20.5, 22, 24, 24.5, and 26 days for mTHPC+laser, mTHPC, EVs-mTHPC, no treatment, laser, Foslip®, and Foslip®+laser respectively). The Kaplan-Meier survival curves of the different groups are shown in FIG. 6. PDT mediated by EVs was able to significantly prolong mice survival in comparison with others PDT-treated group and control groups. The P values of the log-rank test comparisons are shown in table. Non-vectorized mTHPC was the cause of lethal toxicity with 91% of dead mice 4 days after laser illumination. mTHPC vectorization with liposome (Foslip®) and EVs (EVs-mTHPC) allowed to suppress this lethal toxicity, and to prolong survival in comparison with control groups. Biological nanovectorization with EVs permitted a better survival than liposomal vectorization (p=0.005).

In Vivo Toxicology

In this study, the toxicity of mTHPC function of vectorization was systematically investigated in mice following intraperitoneal injection at 0.15 mg/kg. Mice were sacrificed at 72 h after treatment injection and 48 h after illumination. The body weight of the different groups of treatment was similar. Histological assessment was performed to examine tissue damage, mainly liver and kidney which are the most affected organs to iatrogenics. Representative histology results are shown in FIG. 7. In our study, accumulation of mTHPC in the liver after free mTHPC and Foslip®, caused adverse effects including pathological changes in their morphology with nuclear degradation. Histological analysis of the kidney (including the glomeruli) indicate some histopathological changes after IP injection of free mTHPC. These observations were explained with elemental analysis (FIG. 2) which showed relatively low amounts of mTHPC in organs after IP injection of EVs-mTHPC.

### DISCUSSION

We report preclinical evidences to assess the high specificity of biological nanovectorization with EVs-targeted photosensitizer which could enable intraperitoneal photodynamic therapy for peritoneal carcinomatosis of colic and ovarian origin. In the 30 last years, with the development of new types of photosensitizers, PDT has attracted people's interest as a treatment method. The poor prognosis of peritoneal metastasis and recent developments in nanovectorization have generated considerable interest in PDT for this disease.

EVs appears like a natural drug delivery vehicles with negligible immunogenicity at contrary to synthetic nanovectors as liposomes [van Dommelen 2012]. Much better stability and intracellular accumulation was demonstrated for EVs-mTHPC compared to mTHPC liposomal formulation [Millard 18]. mTHPC embedding into EVs prevents PS aggregation, like liposomal nanovectors [Reshetov 2012], with a better tumoral vectorization and slower clearance. For tumor tissue, the mTHPC concentration with liposomal vectorization was the most important at 15 h (0.21±0.07 ng/mg tissue), following by a rapid clearance (at 24 h: 0.06±0.01 ng/mg tissue). Xie et al described too, with a liposomal formulation of mTHPC, a maximal mTHPC concentration of 0.17±0.07 ng/mg at 18 h post injection [Xie15]. At 4 h, we measured an intra tumoral concentration at 0.15±0.04 nm/mg tissue, which corresponds to the data of the literature (0.16±0.024 ng/mg tissue in Svensson analyze [Svensson06]. So we proposed a new generation of PS, with a biological vectorization, which allows a concentration of higher intra-tumor PS, and a more important tumor selectivity after IP injection. The tumor-to-normal tissue ratio allows understanding the high grade morbidity observed both in preclinical and clinical studies. Our analysis yielded a tumor-to-organ ratio of 19 at 4 h, 24 at 15 h, 44 at 24 h and 7 at 48 h, which was significantly higher than Foslip® (3 at 4 h, 5 at 15 h, 2 at 24 h and 6 at 48 h) and higher than literature ratio [Morlet95, Veenhuizen97]. The hydrophobic mTHPC (without vectorization) preferentially accumulated in organs rich in mononuclear phagocytic cells (e.g., liver and spleen) to a higher degree than in other types of tissues. In ovarian model of carcinomatosis, EVs permitted an tumoral vectorization too with a peak at 24 h. A mean tumor-to-normal tissue of 15, which is highest of recent PDT vectorisation in literature [Azaïs16], should permit to illuminate the peritoneal cavity without inducing visceral injuries. These results showed an important ratio regarding liver kidney and bowel (tumor-to-tissue ratio=12, 23 and 25 respectively), indicating that it could be possible to illuminate those organs with appropriate wavelength without risking an hepatic or kidney injury or digestive perforation. However these complications limited the development of PDT yet. We propose an IP injection of PS. In literature, Perry (Perry 1991) and Veenhuizen (Veenhuizen 1997) compared drug uptake after IV and IP injection. Perry (Perry 1991) described a longer tumoral elimination half-time (113.6 h vs 60.6 h) with IP administration, with a lower liver and kidney uptake. Veenhuizen (Veenhuizen 1997) described a higher disseminated tumoral drug uptake after IP administration too (about 20 times that after IV administration).

Effectiveness of PDT in colorectal peritoneal metastasis was knows for more than 30 years (Tochner 1985). Clinical trials showed important side effects (capillary leak syndrome and bowel perforation), mainly explained by low tumor-selectivity of the PS used (first generation mainly) (Pinto 2018). However, in preclinical studies, each new generation of PS permitted to improve tumoral targeting, with less toxicity and better effectiveness. Ascencio (Ascensio 2008), Estevez (Estevez 2010), Mroz (Mroz 2011), Hino (Hino 2013) and Kato (Kato 2017) described carcinomatosis necrosis, until 77% complete response (Ascensio 2008). Our results were inferior to those reported in the literature in 3 different PC models. Ascensio and Estevez [Ascencio 2008, Estevez 2010] found, 24 hours after PDT treatment, a mean necrosis score of 3.5. However, it was not the same animal model (rat vs mouse), not the same tumor model (ovary vs colon) and we have no information about the toxicity of the treatment on the other organs. However, Mroz [Mroz2011] described, after PDT in a mouse model of colorectal PC, a tumoral cytotoxicity by apoptosis and necrosis on the periphery of the nodules whereas it consisted rather in a necrosis in the center. In our study, necrosis and apoptosis concerned the both. Moreover, we first demonstrated the tumoral infiltration with macrophages and lymphocytes after PDT treatment.

Survival advantage of PDT was analyzed by some authors. Tochner [Tochner 1991], first, shown 85% survival at 25 days in PDT group, whereas all mice not receiving HPD-laser treatment died between days 20 and 23. Song et al [Song] shown PDT prolonged survival too. The median follow-up time was 45 days (95% CI, 1.17-88.83 days) in the treatment group versus 15 days (95% CI, 6.68-23; 32 days) and 19 days (95% CI, 13.16-24.84 days) in surgery alone group and surgery+laser without PS group (p=0.008). This prolonged survival was described by Yokoyama too (Yokoyama). Mean survival was compared in three groups: debulking surgery (DS) alone, DS+PDT and DS+PDT and clofibric acid. Survival was significantly longer 35.5 days, 46.3 days and 52.5 days respectively (p<0.005). We demonstrated the impact of PS vectorization on survival by comparing 2 vectors. EVs permitted a prolonged survival after PDT with 30% of survival in this group, whereas others treated mice were died. It notably allowed a superior survival compared to the liposomal formulation (p=0.005). These 2 vectorized formulations permitted to remove PDT lethal toxicity occurred after free mTHPC IP injection. Intraperitoneal vectorized PDT-related toxicity, such as bowel perforation, did not appear in our study. However, mice treated with non-vectorized PDT died 4 days following treatment. Histological analysis shown hepatic and kidney necrosis in this last group. Liver PDT toxicity is described. Tochner first, (Tochner 1991) shown in all treated animals hemosiderin-like deposits mainly in the periportal parenchymal cells and a very mild non-specific reactive hepatitis in which there was mild hyperplasia of Kuffer cells and few lymphohistiocytic aggregates. Perry (Perry 2001) published significant mortality in PDT groups and animals generally died from liver and small bowel necrosis. Griffin (Griffin 2001) didn't note major acute late clinical effects but all treated dogs and one control dog showed transient elevations in the LFTs, AlkP, AST, and ALT. These liver function test anomalies was noted by Guyon too (Guyon 2014).

To conclude, EVs permitted a biological nanovectorization of mTHPC with an important tumoral selectivity. In vivo studies proved that EVs-based PDT was effective for colorectal peritoneal metastasis. It permitted an intra-tumoral cytotoxic effect of PDT by direct and indirect mechanisms. Particularly, we observed an intra-tumor macrophages infiltration after PDT and a lymphocyte infiltration provided by the vesicles. This is the first time to our knowledge that this immunostimulatory effect is analyzed in vivo after vectorization of a PS in a CP model.

### REFERENCES

Throughout this application, various references describe the state of the art to which this invention pertains. The disclosures of these references are hereby incorporated by reference into the present disclosure.

1. An isolated mesenchymal stem cell derived extracellular vesicle loaded with at least one photosensitizer.

2. The isolated mesenchymal stem cell derived extracellular vesicle of claim 1, wherein the at least one photosensitizer is selected from the group consisting of porphyrins, hydroporphyrins, chlorins, bacteriochlorins, purpurins, porphycenes, verdins, cyanines, merocyanines, phthalocyanines, chloroaluminum and phthalocyanines.

3. The isolated mesenchymal stem cell derived extracellular vesicle of claim 1 wherein the at least one photosensitizer is meta-tetra hydroxyphenylchlorin (mTHPC).

4. A population of mesenchymal stem cell derived extracellular vesicles (MSC-EVs) according to claim 1.

5. A method of preparing the population of claim 4 comprising the steps of
i) causing a turbulent flow of a culture medium in a container, wherein the culture medium comprises mesenchymal stem cells adhering to the surface of microcarriers, the microcarriers being in suspension in the culture medium, and wherein the culture medium also comprises an amount of the at least one photosensitizer, and then
ii) collecting the population of mesenchymal stem cell derived extracellular vesicles from the culture medium.

6. (canceled)

7. A method reducing tumor cell growth and/or proliferation in a subject in need thereof comprising the steps of i) administering to the subject a therapeutically effective amount of the population of MSC-EVs of claim 4; and ii) light-activating photosensitizer loaded on the extracellular vesicles to produce cytotoxic species, wherein the cytotoxic species inhibit the tumor cell growth and/or proliferation.

8. A method of treating cancer occurring in body cavity in a subject in need thereof, comprising the steps of i) administering to the body cavity of the subject a therapeutically effective amount of the population of MSC-EVs of claim 4; and ii) light-activating photosensitizer loaded on the MSC-EVs to produce cytotoxic species, wherein the cytotoxic species inhibit tumor cell growth and/or proliferation in the body cavity, thereby treating the cancer.

9. The method of claim 8, wherein the cancer occurring in body cavity is peritoneal carcinomatosis or pleural metastasis.

10. The method of claim 9 wherein the peritoneal carcinomatosis results from a colorectal cancer or an ovarian cancer.

11. The method of claim 9 wherein the peritoneal carcinomatosis is pseudomyxoma peritonei.

12. The method of claim 8 wherein step ii) is performed via coelioscopy, laparoscopy, or thoracoscopy.

13. The method of claim 8 wherein steps i) an ii) are repeated at least 2, 3, 4, or 5 times.

14. A pharmaceutical composition comprising an amount of the population of claim 4.

15. The isolated mesenchymal stem cell derived extracellular vesicle of claim 2, wherein the phthalocyanines are phthalocyanines with metal substituents or phthalocyanines without metal substituents.
